(19) United States
(12) Patent Application Publication
(10) Pub. No.: US 2010/0074881 A1
(43) Pub. Date: Mar. 25, 2010

(54) MULTIPLE NEBULIZER SYSTEMS

(75) Inventors: Richard C. BOUCHER, Durham, NC (US); Michael R. Johnson, Durham, NC (US); Keith A. Johnson, Durham, NC (US); William R. Thelin, Durham, NC (US)

Correspondence Address: OBLON, SPIVAK, MCCLELLAND MAIER & NEUSTADT, L.L.P., 1940 DUKE STREET, ALEXANDRIA, VA, 22314, US

(73) Assignee: PARION SCIENCES, Inc., Durham, NC (US)

(21) Appl. No.: 12/501,654

(22) Filed: Jul. 13, 2009

Related U.S. Application Data
(60) Provisional application No. 61/079,989, filed on Jul. 11, 2008.

(51) Int. Cl. A61K 31/4965; A61P 11/00; A61K 38/46; A61K 31/7036; A61K 38/12; A61K 33/24; A61M 16/12

(52) U.S. Cl. 424/94.6; 514/255.06; 514/40; 514/9; 424/655; 128/203.12

(57) ABSTRACT

A device for administering two or more therapeutic agents simultaneously, comprising two or more nebulizers and a single connector linking the nebulizers to a nebulizer mouthpiece. Also provided is a method of administering two or more therapeutic agents simultaneously, comprising administering the therapeutic agents simultaneously with the device of the present invention to a subject in need thereof.

### CONTINUING APPLICATION INFORMATION

The present application claims benefit to U.S. provisional application Ser. No. 61/079,989, filed on Jul. 11, 2008, and incorporated herein by reference.

### FIELD OF THE INVENTION

The present invention relates to multiple nebulizer technology for the simultaneous delivery of two or more therapeutic agents. The present invention also includes a variety of applications for the co-delivery of two or more therapeutic agents.

### BACKGROUND OF THE INVENTION

The mucosal surfaces at the interface between the environment and the body have evolved a number of “innate defenses”, i.e., protective mechanisms. A principal form of such innate defense is to cleanse these surfaces with liquid. Typically, the quantity of the liquid layer on a mucosal surface reflects the balance between epithelial liquid secretion, often reflecting active anion (Cl⁻ and/or HCO₃⁻) secretion coupled with water (and a cation counter-ion), and epithelial liquid absorption, often reflecting active Na⁺ absorption, coupled with water and counter anion (Cl⁻ and/or HCO₃⁻).

Increasing the protective liquid layer on mucosal surfaces is useful for preventing and/or treating diseases of the lungs. For example, diseases of mucosal surfaces, such as cystic fibrosis, are caused by too little protective liquid on those mucosal surfaces created by an imbalance between secretion (too little) and absorption (relatively too much). The defective salt transport processes that characterize these mucosal dysfunctions reside in the epithelial layer of the mucosal surface. Alternatively, increasing the protective liquid layer accelerates the clearance of inhaled particles from the lungs, including hazardous agents such as bacteria, viruses, or radioactive particles.

One approach to increase the protective liquid layer on mucosal surfaces is to “re-balance” the system by blocking Na⁺ channel and liquid absorption. The epithelial protein that mediates the rate-limiting step of Na⁺ and liquid absorption is the epithelial Na⁺ channel (ENaC). ENaC is positioned on the apical surface of the epithelium, i.e. the mucosal surface-environmental interface. Therefore, to inhibit ENaC mediated Na⁺ and liquid absorption, an ENaC blocker of the amiloride class (which blocks from the extracellular domain of ENaC) must be delivered to the mucosal surface and, importantly, be maintained at this site, to achieve therapeutic utility.

Chronic obstructive pulmonary diseases are characterized by dehydration of airway surfaces and the retention of mucous secretions in the lungs. Examples of such diseases include cystic fibrosis, chronic bronchitis, and primary or secondary ciliary dyskinesia. Such diseases affect approximately 15 million patients in the United States, and are the sixth leading cause of death. Other airway or pulmonary diseases characterized by the accumulation of retained mucous secretions include sinusitis (an inflammation of the paranasal sinuses associated with upper respiratory infection) and pneumonia.

U.S. Pat. No. 5,817,028 to Anderson describes a method for the provocation of air passage narrowing (for evaluating susceptibility to asthma) and/or the induction of sputum in subjects via the inhalation of mannitol. It is suggested that the same technique can be used to induce sputum and promote mucociliary clearance. Substances suggested include sodium chloride, potassium chloride, mannitol and dextrose.

Chronic bronchitis (CB), including the most common lethal genetic form of chronic bronchitis, cystic fibrosis (CF), a disease that reflects the body's failure to clear mucus normally from the lungs, which ultimately produces chronic airways infection. In the normal lung, the primary defense against chronic intrapulmonary airways infection (chronic bronchitis) is mediated by the continuous clearance of mucus from bronchial airway surfaces. This function in health effectively removes from the lung potentially noxious toxins and pathogens. Recent data indicate that the initiating problem, i.e., the “basic defect,” in both CB and CF is the failure to clear mucus from airway surfaces. The failure to clear mucus reflects dehydration of airway surfaces that reflects an imbalance between the amount of liquid and mucin on airway surfaces. This “airway surface liquid” (ASL) is primarily composed of salt and water in proportions similar to plasma (i.e., isotonic). Mucin macromolecules organize into a well defined “mucus layer” which normally traps inhaled bacteria and is transported out of the lung via the actions of cilia which beat in a watery, low viscosity solution termed the “periciliary liquid” (PCL). In the disease state, there is an imbalance in the quantities of mucins (too much) and ASL (too little) on airway surfaces that produces airway surface dehydration. This dehydration leads to mucus concentration, reduction in the lubricant activity of the PCL, and a failure to clear mucus via ciliary activity to the mouth. The reduction in mechanical clearance of mucus from the lung leads to chronic airways inflammation and bacterial colonization of mucus adherent to airway surfaces. It is the chronic retention of bacteria, the failure of local antimicrobial substances to kill mucus-entrapped bacteria on a chronic basis, and the consequent chronic inflammatory responses of the body to this type of surface infection, that lead to the destruction of the lung in CB and CF.

The current afflicted population in the U.S. is 12,000,000 patients with the acquired (primarily from cigarette smoke exposure) form of chronic bronchitis and approximately 30,000 patients with the genetic form, cystic fibrosis. Approximately equal numbers of both populations are present in Europe. In Asia, there is little CF but the incidence of CB is high and, like the rest of the world, is increasing.

There is currently a large, unmet medical need for products that specifically treat CB and CF at the level of the basic defect that cause these diseases. The current therapies for chronic bronchitis and cystic fibrosis focus on treating the symptoms and/or the late effects of these diseases. Thus, for chronic bronchitis, β-agonists, inhaled steroids, anti-cholinergic agents, and oral theophyllines and phosphodiesterase inhibitors are all in development. However, none of these drugs treat effectively the fundamental problem of the failure to clear mucus from the lung. Similarly, in cystic fibrosis, the same spectrum of pharmacologic agents is used. These strategies have been complemented by more recent strategies designed to clear the CF lung of the DNA (“Pulmozyme”; Genentech) that has been deposited in the lung by neutrophils that have futilely attempted to kill the bacteria that grow in adherent mucus masses and through the use of inhaled antibiotics (“TOBI”) designed to augment the lungs' own killing mechanisms to rid the adherent mucus plaques of bacteria. A general principle of the body is that if the initiating lesion is not treated, in this case mucus retention/obstruction, bacterial infections became chronic and increasingly refractory to antimicrobial therapy. Thus, a major unmet therapeutic need for both CB and CF lung diseases is an effective means of re-hydrating airway mucus (i.e., restoring/expanding the volume of the ASL) and promoting its clearance, with bacteria, from the lung.

R. C. Boucher, in U.S. Pat. No. 6,264,975, describes the use of pyrazinoylguanidine sodium channel blockers for hydrating mucosal surfaces. These compounds, typified by the well-known diuretics amiloride, benzamil, and phenamil, are effective. However, these compounds suffer from the significant disadvantage that they are (1) relatively impotent, which is important because the mass of drug that can be inhaled by the lung is limited; (2) rapidly absorbed, which limits the half-life of the drug on the mucosal surface; and (3) are freely dissociable from ENaC. The sum of these disadvantages embodied in these well known diurectics produces compounds with insufficient potency and/or effective half-life on mucosal surfaces to have therapeutic benefit for hydrating mucosal surfaces.

R. C. Boucher, in U.S. Pat. No. 6,926,911, suggests the use of the relatively impotent sodium channel blockers such as amiloride, with osmolytes for the treatment of airway disesases. This combination gives no practical advantage over either treatment alone and is clinically not useful, see Donaldson et al, N Eng J Med2006; 353:241-250. Amiloride was found to block the water permeability of airways and negate the potential benefit of concurrent use of hypertonic saline and amiloride.

Clearly, what is needed are treatments that are more effective at restoring the clearance of mucus from the lungs of patients with CB/CF. The value of these new therapies will be reflected in improvements in the quality and duration of life for both the CF and the CB populations.

Other mucosal surfaces in and on the body exhibit subtle differences in the normal physiology of the protective surface liquids on their surfaces but the pathophysiology of disease reflects a common theme, i.e., too little protective surface liquid. For example, in xerostomia (dry mouth) the oral cavity is depleted of liquid due to a failure of the parotid sublingual and submandibular glands to secrete liquid despite continued Na⁺ (ENaC) transport mediated liquid absorption from the oral cavity. Similarly, keratoconjunctivitis sica (dry eye) is caused by failure of lacrimal glands to secrete liquid in the face of continued Na⁺ dependent liquid absorption on conjunctional surfaces. In rhinosinusitis and otis media, there is an imbalance, as in CB, between mucin secretion and relative ASL depletion. Finally, in the gastrointestinal tract, failure to secrete Cl⁻ (and liquid) in the proximal small intestine, combined with increased Na⁺ (and liquid) absorption in the terminal ileum leads to the distal intestinal obstruction syndrome (DIOS). In older patients excessive Na⁺ (and volume) absorption in the descending colon produces constipation and diverticulitis.

Additionally, it is believed that the sodium channel blockers disclosed herein surprisingly may be used on substantially normal or healthy lung tissue to prevent or reduce the uptake of airborne pathogens and/or to clear the lungs of all or at least a portion of such pathogens. Preferably, the sodium channel blockers will prevent or reduce the viral or bacterial uptake of airborne pathogens. The ability of sodium channel blockers to hydrate mucosal surfaces is believed to function to first hydrate lung mucous secretions, including mucous containing the airborne pathogens to which the human has been subjected, and then facilitate the removal of the lung mucous secretions from the body. By functioning to remove the lung mucous secretions from the body, the sodium channel blocker thus prevents or, at least, reduces the risk of infection from the pathogen(s) inhaled or brought into the body through a bodily airway. Therefore, the prophylactic or therapeutic treatment methods of the present invention may be used in situations where a segment of the population has been, or is believed to have been, exposed to one or more airborne pathogens. The prophylactic or therapeutic treatment methods may additionally be used in situations of ongoing risk of exposure to or infection from airborne pathogens. Such situations may arise due to naturally occurring pathogens or may arise due to a bioterrorism event wherein a segment of the population is intentionally exposed to one or more pathogens. The individuals or portion of the population believed to be at risk from infection can be treated according to the methods disclosed herein. Such treatment preferably will commence at the earliest possible time, either prior to exposure if imminent exposure to a pathogen is anticipated or possible or after the actual or suspected exposure. Typically, the prophylactic treatment methods will be used on humans asymptomatic for the disease for which the human is believed to be at risk. The term “asymptomatic” as used herein means not exhibiting medically recognized symptoms of the disease, not yet suffering from infection or disease from exposure to the airborne pathogens, or not yet testing positive for a disease. The treatment methods may involve post-exposure prophylactic or therapeutic treatment, as needed.

The pathogens which may be protected against by the prophylactic post exposure, rescue and therapeutic treatment methods of the invention include any pathogens which may enter the body through the mouth, nose or nasal airways, thus proceeding into the lungs. Typically, the pathogens will be airborne pathogens, either naturally occurring or by aerosolization. The pathogens may be naturally occurring or may have been introduced into the environment intentionally after aerosolization or other method of introducing the pathogens into the environment. Many pathogens which are not naturally transmitted in the air have been or may be aerosolized for use in bioterrorism.

The multiple nebulizer system is particularly useful to treat diseases or conditions wherein multiple therapies are to be used simultaneously and cannot be formulated together due to incompatible properties such as solubility. This nebulizer system is particularly useful in treating diseases and conditions of the lung. Such diseases and conditions include treating chronic bronchitis, treating bronchiectasis, treating cystic fibrosis, treating sinusitis, promoting mucus clearance in mucosal surfaces, treating esophagitis, treating asthma, treating primary ciliary dyskinesia, treating otitis media, inducing sputum for diagnostic purposes, treating chronic obstructive pulmonary disease, treating emphysema, treating pneumoniatreating rhinosinusitisas well as to administer prophylactic, post-exposure prophylactic, preventive or therapeutic treatments against diseases or conditions caused by pathogens, nuclear fallout, dust, toxic particles and other airborne acts of war or terrorism.

### SUMMARY OF THE INVENTION

The present invention relates to a device for administering two or more therapeutic agents simultaneously, comprising two or more nebulizers and a single connector linking the nebulizers to a nebulizer mouthpiece. The present invention also relates to a method of administering two or more therapeutic agents simultaneously, comprising administering the therapeutic agents simultaneously with the device of the present invention to a subject in need thereof.

### BRIEF DESCRIPTION OF THE DRAWINGS

FIG. 1: Design of dual nebulizer systems. Aerosol generation from each nebulizer can be driven by independent compressors (FIG. 1A) or a single compressor connected to a Y-splitter (FIG. 1B).

FIG. 2: Drug delivery from a dual nebulizer.

### DETAILED DESCRIPTION OF THE INVENTION

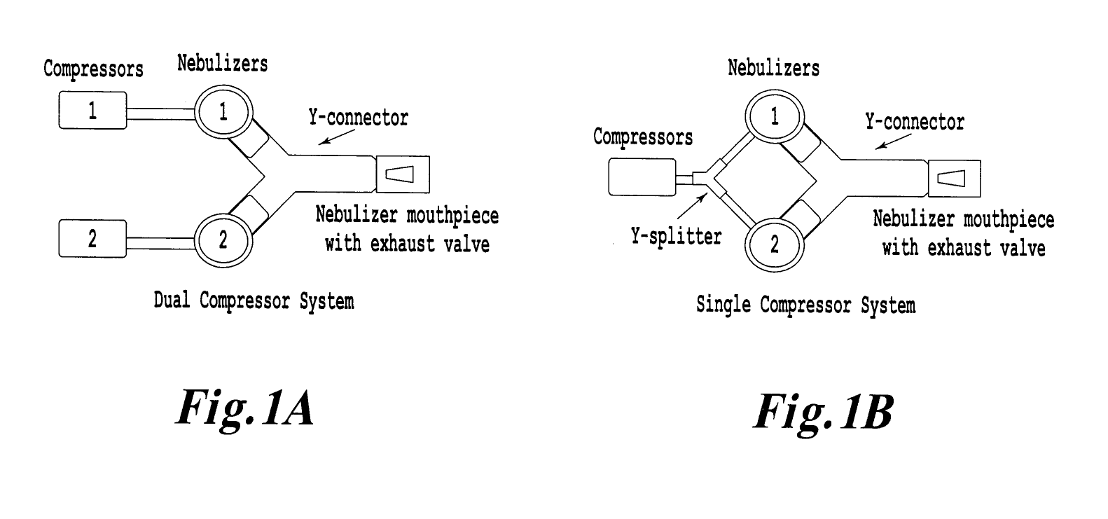

The present invention comprises connecting two or more nebulizer systems through a common connector. FIG. 1 shows the design of a novel dual nebulizer system employing a Y-connector. The dual nebulizer system requires minimal modification of existing units and is composed entirely of commercially available components. Essentially, this system combines the output of two independent nebulizers through a Y-connector. Furthermore, aerosol generation from each nebulizer can be driven by independent compressors (FIG. 1A) or a single compressor connected to a Y-splitter (FIG. 1B).

Thus, in one embodiment, the single connector is a Y-connector. In another embodiment of the invention, the connector is a Y-splitter. These types of connectors are well-known in the art. For example, see A. Berlinski and J. C. Waldrep. J. Aerosol Med. 19, 484-490 (2006), incorporated herein by reference.

In a preferred embodiment of the invention, the device further comprises at least one compressor connected to at least one of the nebulizers. Compressors are also well-known in the art. For example, see P. P Le Brun et al. Pharm. World Sci. 22, 75-81 (2000), incorporated herein by reference.

In another preferred embodiment, the device comprises one compressor connected to two nebulizers. In another embodiment, each compressor is corrected to a different nebulizer.

In another embodiment of the invention, each nebulizer contains at least one therapeutic agent.

In yet another embodiment, each nebulizer contains a different therapeutic agent, and the therapeutic agents are incompatible in a single formulation. Example of such therapeutic agents are DNase, Tobi, colistin, a beta agonist, a P₂Y₂ agonist, sodium chlorochromate, an osmolyte, and ENaC blockers. Examples of such compounds are well-known in the art, see M. T. Clunes and R. C. Boucher, Current Opin. Pharmacol. 8, 292-299 (2008) and C. Frerichs and A. Smyth, Expert Opin. Pharmacother. 10, 1191-1202 (2009), incorporated herein by reference.

In a particularly preferred embodiment, one therapeutic agent is compound PS552-02. In an especially preferred embodiment, one therapeutic agent is compound PS552-02 and another therapeutic agent is hypertonic saline. Compound PS552-02 is represented by the following formula:

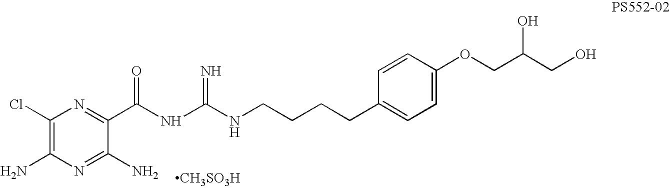

The present invention also includes a method of administering two or more therapeutic agents simultaneously, comprising administering the therapeutic agents simultaneously with the device discussed above to a subject in need thereof.

Suitable subjects include humans and animals.

In one embodiment, the subject is suffering from a pulmonary disorder and the therapeutic agents are for treating the pulmonary disorder. Pulmonary disorders include CF and CB.

In another embodiment, the subject is suffering from pulmonary exposure to a hazardous airborne agent and the therapeutic agents are for enhancing clearance of the inhaled hazardous agent. Inhaled hazardous agents include pathogens such as bacteria and viruses or radioactive particles.

In yet another embodiment, the subject will administer the therapeutic agent as prophylaxis (preventive) prior to exposure to inhaled hazardous agents. Inhaled hazardous agents include pathogens such as bacteria and viruses or radioactive particles.

In one embodiment of the invention, the therapeutic agents are incompatible in a single formulation. In a preferred embodiment, the therapeutic agents are selected from the group consisting of DNase, Tobi, colistin, a beta agonist, a P₂Y₂ agonist, sodium chlorochromate, an osmolyte, and ENaC blockers.

It is an object of the present invention to provide delivery of treatments comprising the use of osmolytes together with sodium channel blockers that are more potent, more specific, and/or absorbed less rapidly from mucosal surfaces, and/or are less reversible as compared to compounds such as amiloride, benzamil, and phenamil.

It is another aspect of the present invention to provide delivery of treatments using sodium channel blockers that are more potent and/or absorbed less rapidly and/or exhibit less reversibility, as compared to compounds such as amiloride, benzamil, and phenamil when administered with an osmotic enhancer. Therefore, such sodium channel blockers when used in conjunction with osmolytes will give a prolonged pharmacodynamic half-life on mucosal surfaces as compared to either compound used alone.

It is another object of the present invention to provide delivery of treatments using sodium channel blockers and osmolytes together which are absorbed less rapidly from mucosal surfaces, especially airway surfaces, as compared to compounds such as amiloride, benzamil, and phenamil.

It is another object of the invention to provide delivery of compositions which contain sodium channel blockers and osmolytes.

The objects of the invention may be accomplished with a method of treating a disease ameliorated by increased mucociliary clearance and mucosal hydration comprising administering an effective amount of a sodium channel blocker as defined herein and an osmolyte to a subject to a subject in need of increased mucociliary clearance and mucosal hydration.

The objects of the invention may also be accomplished with composition, comprising a sodium channel blocker as defined herein and an osmotically active compound.

A more complete appreciation of the invention and many of the attendant advantages thereof will be readily obtained as the same becomes better understood by reference to the following detailed description of the invention.

The term “sodium channel blocker as defined herein” as used herein refers to the sodium channel blockers described in U.S. patent application Ser. No. 10/076,551 (see pages 4-52), filed Feb. 19, 2002; U.S. Pat. No. 6,858,614 (see column 3, line 47 to column 29, line 64); WO 2004/073629 (see pages 5-107); U.S. patent application Ser. No. 10/367,947 (see pages 5-45, filed Feb. 19, 2003; U.S. Pat. No. 6,903,105 (see columns 4-33); U.S. patent application Ser. No. 10/920,410 (see pages 5-80), filed Aug. 18, 2004; U.S. Pat. No. 7,064,129 (see columns 4-76), U.S. patent application Ser. No. 10/920,391 (see pages 5-91), filed Aug. 18, 2004, WO 2006/022935 (see pages 5-91), WO 2006/023573 (see pages 5-55), WO 2006/023617 (see pages 5-56), U.S. patent application Ser. No. 10/920,353 (seepages 5-68), filed Aug. 18, 2004; U.S. patent application Ser. No. 10/920, 418 (see pages 5-72), filed Aug. 18, 2004; and U.S. provisional application Ser. Nos. 60/495,725, 60/602,327, 60/495,720, 60/602,312, and 60/495,712, and U.S. patent application Ser. No. 11/195,758, each of which is incorporated herein by reference. All racemates, enantiomers, diastereomers, tautomers, polymorphs and pseudopolymorphs, salts and racemic mixtures of the sodium channel blockers are embraced by the present invention. The specific examples of sodium channel blockers described in those applications and patents are explicitly incorporated herein by reference. The sodium channel blockers may be synthesized as described in those applications and patents.

Thus, the sodium channel blockers useful in the present invention are represented by formula (I):

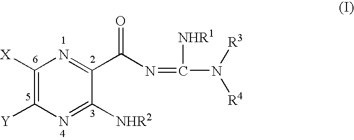

Detailed descriptions and specific examples of compounds represented by formula (I) are found in the references cited above.

Specific examples of sodium channel blockers that may be used in the present invention include:

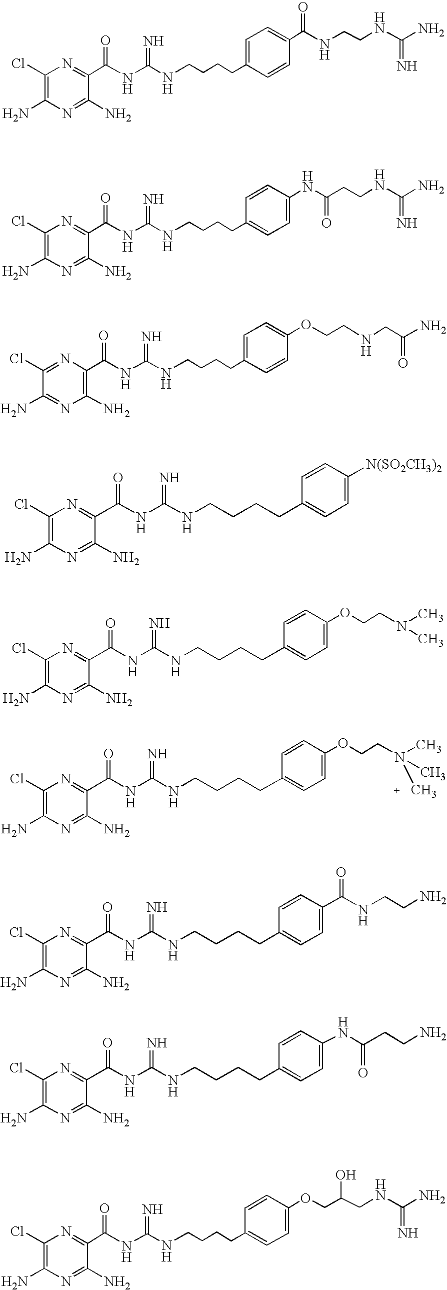

The compounds of formula (I) may be synthesized according to procedures known in the art. A representative synthetic procedure is shown in the scheme below:

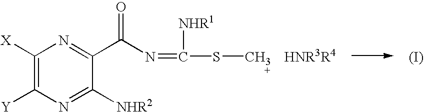

These procedures are described in, for example, E. J. Cragoe, “The Synthesis of Amiloride and Its Analogs” (Chapter 3) in Amiloride and Its Analogs, pp. 25-36, incorporated herein by reference. Other methods of preparing the compounds are described in, for example, U.S. Pat. No. 3,313,813, incorporated herein by reference. See in particular Methods A, B, C, and D described in U.S. Pat. No. 3,313,813. Several assays may be used to characterize the compounds of the present invention. Representative assays are discussed below.

Without being limited to any particular theory, it is believed that sodium channel blockers of the present invention block epithelial sodium channels present in mucosal surfaces the sodium channel blocker, described herein reduce the absorption of salt and water by the mucosal surfaces. This effect increases the volume of protective liquids on mucosal surfaces, rebalances the system, and thus treats disease. This effect is enhanced when used in combination with osmolytes.

Active osmolytes of the present invention are molecules or compounds that are osmotically active (i.e., are “osmolytes”). “Osmotically active” compounds of the present invention are membrane-impermeable (i.e., essentially non-absorbable) on the airway or pulmonary epithelial surface. The terms “airway surface” and “pulmonary surface,” as used herein, include pulmonary airway surfaces such as the bronchi and bronchioles, alveolar surfaces, and nasal and sinus surfaces. Active compounds of the present invention may be ionic osmolytes (i.e., salts), or may be non-ionic osmolytes (i.e., sugars, sugar alcohols, and organic osmolytes). It is specifically intended that both racemic forms of the active compounds that are racemic in nature are included in the group of active compounds that are useful in the present invention. It is to be noted that all racemates, enantiomers, diastereomers, tautomers, polymorphs and pseudopolymorphs and racemic mixtures of the osmotically active compounds are embraced by the present invention.

Active osmolytes useful in the present invention that are ionic osmolytes include any salt of a pharmaceutically acceptable anion and a pharmaceutically acceptable cation. Preferably, either (or both) of the anion and cation are non-absorbable (i.e., osmotically active and not subject to rapid active transport) in relation to the airway surfaces to which they are administered. Such compounds include but are not limited to anions and cations that are contained in FDA approved commercially marketed salts, see, e.g., Remington: The Science and Practice of Pharmacy, Vol. II, pg. 1457 (19^{th} Ed. 1995), incorporated herein by reference, and can be used in any combination including their conventional combinations.

Pharmaceutically acceptable osmotically active anions that can be used to carry out the present invention include, but are not limited to, acetate, benzenesulfonate, benzoate, bicarbonate, bitartrate, bromide, calcium edetate, camsylate (camphorsulfonate), carbonate, chloride, citrate, dihydrochloride, edetate, edisylate (1,2-ethanedisulfonate), estolate (lauryl sulfate), esylate (1,2-ethanedisulfonate), fumarate, gluceptate, gluconate, glutamate, glycollylarsanilate (p-glycollamidophenylarsonate), hexylresorcinate, hydrabamine (N,N′-Di(dehydroabietyl)ethylenediamine), hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, nitrte, pamoate (embonate), pantothenate, phosphate or diphosphate, polygalacturonate, salicylate, stearate, subacetate, succinate, sulfate, tannate, tartrate, teoclate (8-chlorotheophyllinate), triethiodide, bicarbonate, etc. Particularly preferred anions include chloride sulfate, nitrate, gluconate, iodide, bicarbonate, bromide, and phosphate.

Pharmaceutically acceptable cations that can be used to carry out the present invention include, but are not limited to, organic cations such as benzathine (N,N′-dibenzylethylenediamine), chloroprocaine, choline, diethanolamine, ethylenediamine, meglumine (N-methyl D-glucamine), procaine, D-lysine, L-lysine, D-arginine, L-arginine, triethylammonium, N-methyl D-glycerol, and the like. Particularly preferred organic cations are 3-carbon, 4-carbon, 5-carbon and 6-carbon organic cations. Metallic cations useful in the practice of the present invention include but are not limited to aluminum, calcium, lithium, magnesium, potassium, sodium, zinc, iron, ammonium, and the like. Particularly preferred cations include sodium, potassium, choline, lithium, meglumine, D-lysine, ammonium, magnesium, and calcium.

Specific examples of osmotically active salts that may be used with the sodium channel blockers described herein to carry out the present invention include, but are not limited to, sodium chloride, potassium chloride, choline chloride, choline iodide, lithium chloride, meglumine chloride, L-lysine chloride, D-lysine chloride, ammonium chloride, potassium sulfate, potassium nitrate, potassium gluconate, potassium iodide, ferric chloride, ferrous chloride, potassium bromide, etc. Either a single salt or a combination of different osmotically active salts may be used to carry out the present invention. Combinations of different salts are preferred. When different salts are used, one of the anion or cation may be the same among the differing salts.

Osmotically active compounds of the present invention also include non-ionic osmolytes such as sugars, sugar-alcohols, and organic osmolytes. Sugars and sugar-alcohols useful in the practice of the present invention include but are not limited to 3-carbon sugars (e.g., glycerol, dihydroxyacetone); 4-carbon sugars (e.g., both the D and L forms of erythrose, threose, and erythrulose); 5-carbon sugars (e.g., both the D and L forms of ribose, arabinose, xylose, lyxose, psicose, fructose, sorbose, and tagatose); and 6-carbon sugars (e.g., both the D and L forms of altose, allose, glucose, mannose, gulose, idose, galactose, and talose, and the D and L forms of allo-heptulose, allo-hepulose, gluco-heptulose, manno-heptulose, gulo-heptulose, ido-heptulose, galacto-heptulose, talo-heptulose). Additional sugars useful in the practice of the present invention include raffinose, raffinose series oligosaccharides, and stachyose. Both the D and L forms of the reduced form of each sugar/sugar alcohol useful in the present invention are also active compounds within the scope of the invention. For example, glucose, when reduced, becomes sorbitol; within the scope of the invention, sorbitol and other reduced forms of sugar/sugar alcohols (e.g., mannitol, dulcitol, arabitol) are accordingly active compounds of the present invention.

Osmotically active compounds of the present invention additionally include the family of non-ionic osmolytes termed “organic osmolytes.” The term “organic osmolytes” is generally used to refer to molecules used to control intracellular osmolality in the kidney. See e.g., J. S. Handler et al., Comp. Biochem. Physiol, 117, 301-306 (1997); M. Burg, Am. J. Physiol. 268, F983-F996 (1995), each incorporated herein by reference. Although the inventor does not wish to be bound to any particular theory of the invention, it appears that these organic osmolytes are useful in controlling extracellular volume on the airway/pulmonary surface. Organic osmolytes useful as active compounds in the present invention include but are not limited to three major classes of compounds: polyols (polyhydric alcohols), methylamines, and amino acids. The polyol organic osmolytes considered useful in the practice of this invention include, but are not limited to, inositol, myo-inositol, and sorbitol. The methylamine organic osmolytes useful in the practice of the invention include, but are not limited to, choline, betaine, carnitine (L-, D- and DL forms), phosphorylcholine, lyso-phosphorylcholine, glycerophosphorylcholine, creatine, and creatine phosphate. The amino acid organic osmolytes of the invention include, but are not limited to, the D- and L-forms of glycine, alanine, glutamine, glutamate, aspartate, proline and taurine. Additional osmolytes useful in the practice of the invention include tihulose and sarcosine. Mammalian organic osmolytes are preferred, with human organic osmolytes being most preferred. However, certain organic osmolytes are of bacterial, yeast, and marine animal origin, and these compounds are also useful active compounds within the scope of the present invention.

Under certain circumstances, an osmolyte precursor may be administered to the subject; accordingly, these compounds are also useful in the practice of the invention. The term “osmolyte precursor” as used herein refers to a compound which is converted into an osmolyte by a metabolic step, either catabolic or anabolic. The osmolyte precursors of this invention include, but are not limited to, glucose, glucose polymers, glycerol, choline, phosphatidylcholine, lyso-phosphatidylcholine and inorganic phosphates, which are precursors of polyols and methylamines. Precursors of amino acid osmolytes within the scope of this invention include proteins, peptides, and polyamino acids, which are hydrolyzed to yield osmolyte amino acids, and metabolic precursors which can be converted into osmolyte amino acids by a metabolic step such as transamination. For example, a precursor of the amino acid glutamine is poly-L-glutamine, and a precursor of glutamate is poly-L-glutamic acid.

Also intended within the scope of this invention are chemically modified osmolytes or osmolyte precursors. Such chemical modifications involve linking to the osmolyte (or precursor) an additional chemical group which alters or enhances the effect of the osmolyte or osmolyte precursor (e.g., inhibits degradation of the osmolyte molecule). Such chemical modifications have been utilized with drugs or prodrugs and are known in the art. (See, for example, U.S. Pat. Nos. 4,479,932 and 4,540,564; Shek, E. et al., J. Med. Chem. 19:113-117 (1976); Bodor, N. et al., J. Pharm. Sci. 67:1045-1050 (1978); Bodor, N. et al., J. Med. Chem. 26:313-318 (1983); Bodor, N. et al., J. Pharm. Sci. 75:29-35 (1986), each incorporated herein by reference.

In general, osmotically active compounds of the present invention (both ionic and non-ionic) that do not promote, or in fact deter or retard bacterial growth are preferred.

The active compounds, methods and compositions of the present invention are useful as therapeutics for the treatment of chronic obstructive airway or pulmonary disease in subjects in need of such treatment. The active compounds, compositions and methods described herein may also be used to induce the production of a sputum or mucous sample in a patient. Additionally, the active compounds, compositions and methods described herein can be used for the lavage of the lungs and/or airways of a patient. The active compounds and compositions described herein may also be administered with other active agents that are to be introduced into airways of a subject, and in fact may function as vehicles or carriers for the other active agents.

Suitable subjects to be treated according to the present invention include both avian and mammalian subjects, preferably mammalian. Any mammalian subject in need of being treated according to the present invention is suitable, including dogs, cats and other animals for veterinary purposes. Human subjects are preferred. Human subjects of both genders and at any stage of development (i.e., neonate, infant, juvenile, adolescent, adult) can be treated according to the present invention. Preferred subjects include those humans afflicted with a chronic obstructive airway or pulmonary disease, including but not limited to cystic fibrosis, chronic bronchitis, emphysema, primary and secondary ciliary dyskinesia, sinusitis, and pneumonia. Human subjects afflicted with cystic fibrosis are particularly preferred.

Aerosols of liquid particles comprising the active compound may be produced by any suitable means, such as with a pressure-driven aerosol nebulizer (L C Star) or an ultrasonic nebulizer (Pari eFlow). For example, see U.S. Pat. No. 4,501,729, incorporated herein by reference. Nebulizers are commercially available devices which transform solutions or suspensions of the active ingredient into a therapeutic aerosol mist either by means of acceleration of compressed gas, typically air or oxygen, through a narrow venturi orifice, by means of ultrasonic agitation or by means of a vibrating porous plate. Suitable formulations for use in nebulizers consist of the active ingredient in a liquid carrier, the active ingredient comprising up to 40% w/w of the formulation, but preferably less than 20% w/w. The carrier is typically water (and most preferably sterile, pyrogen-free water), a dilute aqueous alcoholic solution or propylene glycol. Perfluorocarbon carriers may also be used. Optional additives include preservatives if the formulation is not made sterile, for example, methyl hydroxybenzoate, antioxidants, flavoring agents, volatile oils, buffering agents and surfactants.

The dosage of the sodium channel blockers and osmotically active compounds disclosed herein will vary depending on the condition being treated and the state of the subject, but generally may be from about 0.1 or 1 to about 30, 50, or 100 milliosmoles of the osmolyte, deposited on the airway surfaces. The daily dose may be divided among one or several unit dose administrations. The dosage of the sodium channel blockers compound will vary depending on the condition being treated and the state of the subject, but generally may be an amount sufficient to achieve dissolved concentrations of active compound on the nasal airway surfaces of the subject from about 10⁻⁹, 10⁻⁸, 10⁻⁷ to about 10⁻³, 10⁻², or 10⁻¹ moles/liter, and more preferably from about 10⁻⁷ to about 10⁻⁴ moles/liter. Depending upon the solubility of the particular formulation of active compound administered, the daily dose may be divided among one or several unit dose administrations. The daily dose by weight may range from about 0.01, 0.03, 0.1, 0.5 or 1.0 to 10 or 20 milligrams of active agent particles for a human subject, depending upon the age and condition of the subject. A currently preferred unit dose is about 0.5 milligrams of active agent given at a regimen of 2-10 administrations per day. The dosage may be provided as a prepackaged unit by any suitable means (e.g., encapsulating a gelatin capsule).

Other pharmacologically (e.g., bronchodilators) active agents (“third agents”) may be administered concurrently to the subject in need thereof with the sodium channel blockers and osmotically active compounds of the present invention

In particular, bronchodilators may be administered concurrently with the sodium channel blockers and osmotically active compounds of the present invention. Bronchodilators that can be used in the practice of the present invention include, but are not limited to, β-adrenergic agonists including but not limited to epinephrine, isoproterenol, fenoterol, albutereol, terbutaline, pirbuterol, bitolterol, metaproterenol, isoetharine, salmeterol, xinafoate, as well as anticholinergic agents including but not limited to ipratropium bromide, as well as compounds such as theophylline and aminophylline. These compounds may be administered in accordance with known techniques, either prior to or concurrently with the active compounds described herein.

Other active ingredients (“third agents”) that may be administered with the sodium channel blockers and osmotically active compounds of the present invention include ion transport modulators and other active agents known to be useful in the treatment of the subject afflicted with a chronic obstructive pulmonary disease (e.g., DNase, antibiotics, disulfhydryl reducing compounds such as N-acetylcystene, etc.).

Ion transport modulators that can be administered as active agents along with the active compounds of the present invention herein include, purinoceptor (particularly P2Y2) receptor agonists such as UTP, UTP-γ-S, dinucleotide P2Y2 receptor agonists, and β-agonists.

The compounds of the present invention may also be used in conjunction with a P2Y2 receptor agonist or a pharmaceutically acceptable salt thereof (also sometimes referred to as an “active agent” herein). The composition may further comprise a P2Y2 receptor agonist or a pharmaceutically acceptable salt thereof (also sometimes referred to as an “active agent” herein). The P2Y2 receptor agonist is typically included in an amount effective to stimulate chloride and water secretion by airway surfaces, particularly nasal airway surfaces. Suitable P2Y2 receptor agonists are described in columns 9-10 of U.S. Pat. No. 6,264,975, U.S. Pat. No. 5,656,256, and U.S. Pat. No. 5,292,498, each of which is incorporated herein by reference.

Other active ingredients that can be administered in combination with the formulations described herein include nucleic acids or oligonucleotides; viral gene transfer vectors (including adenovirus, adeno-associated virus, and retrovirus gene transfer vectors); enzymes; and hormone drugs or physiologically active proteins or peptides such as insulin, somatostatin, oxytocin, desmopressin, leutinizing hormone releasing hormone, nafarelin, leuprolide, adrenocorticotrophic hormone, secretin, glucagon, calcitonin, growth hormone releasing hormone, growth hormone, etc. Enzyme drugs that may be used to carry out the present invention, include but are not limited to DNAse (for the treatment of, e.g., cystic fibrosis), α₁-antitrypsin (e.g., to inhibit elastase in the treatment of emphysema), etc. Suitable anti-inflammatory agents, including steroids, for use in the methods of the present invention include, but are not limited to, beclomethasone dipropionate, prednisone, flunisolone, dexamethasone, prednisolone, cortisone, theophylline, albuterol, cromolyn sodium, epinephrine, flunisolide, terbutaline sulfate, alpha-tocopherol (Vitamin E), dipalmitoylphosphatidylcholine, salmeterol and fluticasone dipropionate. Examples of antibiotics that may be employed include, but are not limited to tetracycline, choramphenicol, aminoglycosides, for example, tobramycin, beta-lactams, for example ampicillin, cephalosporins, erythromycin and derivatives thereof, clindamycin, phosphonic acid antibiotics, for example, fosfomycin, and the like. The antibiotics that may be employed may be used in combination, for example tobramycin and fosfomycin. Suitable anti-viral agents include acyclovir, ribavirin, ganciclovir and foscarnet. Suitable anti-neoplastic agents include, but are not limited to, etoposid, taxol, and cisplatin. Antihistamines include, but are not limited to, diphenhydramine and ranitadine. Anti-Pneumocystis carinii pneumonia drugs such as pentamidine and analogs thereof may also be used. Anti-tuberculosis drugs such as rifampin, erythromycin, chlorerythromycin, etc. Chelators of divalent cations (e.g., EGTA, EDTA), expectorants, and other agents useful in the loosening of mucous secretions (e.g., n-acetyl-L-cysteine) may also be administered as desired in the practice of the present invention.

The present invention is particularly useful for chronic treatments: that is, treatments wherein the administration is repeated two or more times in close proximity to one another, so that the multiple treatments achieve a combined therapeutic effect. For example, the administration may be carried out two, three, four, five, six or seven times a week, on separate days throughout the week. The treatment may be carried out for a period of two, four, or six days or more; daily for two or four weeks or more; daily for two or four months or more, etc. For example, the administering step may be carried out three, four, five or six times a day for the duration of the condition being treated, with chronic conditions receiving chronic treatments.

Solid or liquid particulate pharmaceutical formulations containing active compounds of the present invention should include particles of respirable size: that is, particles of a size sufficiently small to pass through the mouth and larynx upon inhalation and into the bronchi, bronchioles, and (if necessary) the alveoli of the lungs. The bronchioles are a particularly preferred target for delivery to the airway surfaces. In general, particles ranging from about 1 to 5 or 6 microns in size (more particularly, less than about 4.7 microns in size) are respirable. In a preferred embodiment, the geometric standard deviation of the particle size is about 1.7 or smaller. Particles of non-respirable size which are included in the aerosol tend to be deposited in the throat and swallowed, and the quantity of non-respirable particles in the aerosol is preferably minimized. For nasal administration, a particle size in the range of 10-500 μm is preferred to ensure retention in the nasal cavity.

The present invention also provides methods of treatment that take advantage of the properties of the sodium channel blockers and osmotically active compounds discussed above. Thus, subjects that may be treated by the methods of the present invention include, but are not limited to, patients afflicted with cystic fibrosis, primary ciliary dyskinesia, bronchiectasis, chronic bronchitis, chronic obstructive airway disease, artificially ventilated patients, patients with acute pneumonia, etc.

The sodium channel blockers and osmotically active compounds of the present invention are also useful for treating airborne infections. Examples of airborne infections include, for example, RSV. The sodium channel blockers and osmotically active compounds of the present invention are also useful for treating an anthrax infection. The present invention relates to the use of sodium channel blockers and osmotically active compounds of the present invention for prophylactic, post-exposure prophylactic, preventive or therapeutic treatment against diseases or conditions caused by pathogens. In a preferred embodiment, the present invention relates to the use of sodium channel blockers and osmotically active compounds for prophylactic, post-exposure prophylactic, preventive or therapeutic treatment against diseases or conditions caused by pathogens which may be used in bioterrorism.

In recent years, a variety of research programs and biodefense measures have been put into place to deal with concerns about the use of biological agents in acts of terrorism. These measures are intended to address concerns regarding bioterrorism or the use of microorganisms or biological toxins to kill people, spread fear, and disrupt society. For example, the National Institute of Allergy and Infectious Diseases (NIAID) has developed a Strategic Plan for Biodefense Research which outlines plans for addressing research needs in the broad area of bioterrorism and emerging and reemerging infectious diseases. According to the plan, the deliberate exposure of the civilian population of the United States to Bacillus anthracis spores revealed a gap in the nation's overall preparedness against bioterrorism. Moreover, the report details that these attacks uncovered an unmet need for tests to rapidly diagnose, vaccines and immunotherapies to prevent, and drugs and biologics to cure disease caused by agents of bioterrorism.

Much of the focus of the various research efforts has been directed to studying the biology of the pathogens identified as potentially dangerous as bioterrorism agents, studying the host response against such agents, developing vaccines against infectious diseases, evaluating the therapeutics currently available and under investigation against such agents, and developing diagnostics to identify signs and symptoms of threatening agents. Such efforts are laudable but, given the large number of pathogens which have been identified as potentially available for bioterrorism, these efforts have not yet been able to provide satisfactory responses for all possible bioterrorism threats. Additionally, many of the pathogens identified as potentially dangerous as agents of bioterrorism do not provide adequate economic incentives for the development of therapeutic or preventive measures by industry. Moreover, even if preventive measures such as vaccines were available for each pathogen which may be used in bioterrorism, the cost of administering all such vaccines to the general population is prohibitive.

Until convenient and effective treatments are available against every bioterrorism threat, there exists a strong need for preventative, prophylactic or therapeutic treatments which can prevent or reduce the risk of infection from pathogenic agents.

The present invention provides such methods of prophylactic treatment. In one aspect, a prophylactic treatment method is provided comprising administering a prophylactically effective amount of a sodium channel blocker and an osmolyte to an individual in need of prophylactic treatment against infection from one or more airborne pathogens and other inhaled particles. A particular example of an airborne pathogen is anthrax.

The term “inhaled particles” as used herein refers to pathogens, radionuclides, and dust. The term “pathogens” as used herein refers to viruses and bacteria which includes, but is not limited to, Bacillus anthracis (anthrax), Clostridium botulinum (botulism), Yersinia pestis (plague), Variola major (smallpox) and other pox viruses, Francisella tularensis (tularemia), Viral hemorrhagic fevers, Arenaviruses, LCM (lymphocytic choriomeningitis), Junin virus, Machupo virus, Guanarite virus, Lassa Fever, Bunyaviruses, Hantavirus, Rift Valley Fever, Flaviviruses, Dengue, Filoviruses, Ebola Marburg, Burkholderia pseudomallei (melioidosis), Coxiella burnetii (Q fever), Brucella species (brucellosis), Burkholderia mallei (glanders), Ricin toxin from Ricinus communis, Epsilon toxin of Clostridium perfringens, Staphylococcal enterotoxin B, Typhus fever (Rickettsia prowazekii), Food and water-borne pathogens bacteria: Diarrheagenic Escherichia coli, Pathogenic vibrios, Shigella species, Salmonella species, Listeria monocytogenes, campylobacter jejuni, Yersinia enterocolitica; Viruses Caliciviruses, Hepatitis A; Protozoa Cryptosporidium parvum, Cyclospora cayatenensis, Giardia lamblia, Entamoeba histolytica, Toxoplasma, Microsporidia, and Additional viral encephalitides West Nile virus, LaCrosse, California encephalitis, Venezuelan equine encephalitis, Eastern equine encephalitis, Western equine encephalitis, Japanese encephalitis virus, Kyasanur forest virus, Nipah virus and additional hantaviruses, tickborne hemorrhagic fever viruses such as Crimean Congo hemorrhagic fever virus, tickborne encephalitis viruses, yellow fever, multi-drug resistant tuberculosis, influenza, other rickettsias and rabies. The term “radionuclide” as used herein refers to viruses and bacteria which includes, but is not limited to, radioactive isotopes including ⁹⁰Sr, ₁₃₇Cs, ⁶⁰Co, ^{238,239}Pu, ²⁴¹Am, ²⁵²Cf, ²²⁶Ra, ¹⁹²Ir, and ²¹⁰Po which are isotopes of concern for use in a radiological dispersion device (RDD).

In another aspect, a prophylactic treatment method is provided for reducing the risk of infection from an airborne pathogen which can cause a disease in a human, said method comprising administering an effective amount of a sodium channel blocker an osmolyte to the lungs of the human who may be at risk of infection from the airborne pathogen but is asymptomatic for the disease, wherein the effective amount of a sodium channel blocker and osmolye are sufficient to reduce the risk of infection in the human. A particular example of an airborne pathogen is anthrax.

In another aspect, a post-exposure prophylactic treatment or therapeutic treatment method is provided for treating infection from an airborne pathogen comprising administering an effective amount of a sodium channel blocker and an osmolyte to the lungs of an individual in need of such treatment against infection from an airborne pathogen. The pathogens which may be protected against by the prophylactic post exposure, rescue and therapeutic treatment methods of the invention include any pathogens which may enter the body through the mouth, nose or nasal airways, thus proceeding into the lungs. Typically, the pathogens will be airborne pathogens, either naturally occurring or by aerosolization. The pathogens may be naturally occurring or may have been introduced into the environment intentionally after aerosolization or other method of introducing the pathogens into the environment. Many pathogens which are not naturally transmitted in the air have been or may be aerosolized for use in bioterrorism. The hazardous agents pathogens for which the treatment of the invention may be useful includes, but is not limited to, category A, B and C priority pathogens as set forth by the NIAID. These categories correspond generally to the lists compiled by the Centers for Disease Control and Prevention (CDC). As set up by the CDC, Category A agents are those that can be easily disseminated or transmitted person-to-person, cause high mortality, with potential for major public health impact. Pathogens in Category A agents include Bacillus anthracis (anthrax), Clostridium botulinum (botulism), Yersinia pestis (plague), Variola major (smallpox) and other pox viruses, Francisella tularensis (tularemia), Viral hemorrhagic fevers, Arenaviruses, LCM (lymphocytic choriomeningitis), Junin virus, Machupo virus, Guanarite virus, Lassa Fever, Bunyaviruses, Hantavirus, Rift Valley Fever, Flaviviruses, Dengue, Filoviruses, Ebola Marburg. Category B agents are next in priority and include those that are moderately easy to disseminate and cause moderate morbidity and low mortality. Category B agents include Burkholderia pseudomallei (melioidosis), Coxiella burnetii (Q fever), Brucella species (brucellosis), Burkholderia mallei (glanders), Ricin toxin from Ricinus communis, Epsilon toxin of Clostridium perfringens, Staphylococcal enterotoxin B, Typhus fever (Rickettsia prowazekii), Food and water-borne pathogens bacteria: Diarrheagenic Escherichia coli, Pathogenic vibrios, Shigella species, Salmonella species, Listeria monocytogenes, campylobacter jejuni, Yersinia enterocolitica; Viruses Caliciviruses, Hepatitis A; Protozoa Cryptosporidium parvum, Cyclospora cayatenensis, Giardia lamblia, Entamoeba histolytica, Toxoplasma, Microsporidia, and Additional viral encephalitides West Nile virus, LaCrosse, California encephalitis, Venezuelan equine encephalitis, Eastern equine encephalitis, Western equine encephalitis, Japanese encephalitis virus and Kyasanur forest virus. Category C consists of emerging pathogens that could be engineered for mass dissemination in the future because of their availability, ease of production and dissemination and potential for high morbidity and mortality. Category C agents include emerging infectious disease threats such as Nipah virus and additional hantaviruses, tickborne hemorrhagic fever viruses such as Crimean Congo hemorrhagic fever virus, tickborne encephalitis viruses, yellow fever, multi-drug resistant tuberculosis, influenza, other rickettsias and rabies. Furthermore, additional pathogens which may be protected against or the infection risk thereby reduced include influenza viruses, rhinoviruses, adenoviruses and respiratory syncytial viruses, and the like. A further pathogen which may be protected against is the coronavirus which is believed to cause severe acute respiratory syndrome (SARS).

The present invention is concerned primarily with the treatment of human subjects, but may also be employed for the treatment of other mammalian subjects, such as dogs and cats, for veterinary purposes.

As discussed above, the compounds used to prepare the compositions of the present invention may be in the form of a pharmaceutically acceptable free base. Because the free base of the compound is generally less soluble in aqueous solutions than the salt, free base compositions are employed to provide more sustained release of active agent to the lungs. An active agent present in the lungs in particulate form which has not dissolved into solution is not available to induce a physiological response, but serves as a depot of bioavailable drug which gradually dissolves into solution.

Another aspect of the present invention is a pharmaceutical composition, comprising a sodium channel blocker in a pharmaceutically acceptable carrier (e.g., an aqueous carrier solution). In general, the sodium channel blocker is included in the composition in an amount effective to inhibit the reabsorption of water by mucosal surfaces.

The compounds of the present invention may also be used in conjunction with a P2Y2 receptor agonist or a pharmaceutically acceptable salt thereof (also sometimes referred to as an “active agent” herein). The composition may further comprise a P2Y2 receptor agonist or a pharmaceutically acceptable salt thereof (also sometimes referred to as an “active agent” herein). The P2Y2 receptor agonist is typically included in an amount effective to stimulate chloride and water secretion by airway surfaces, particularly nasal airway surfaces. Suitable P2Y2 receptor agonists are described in columns 9-10 of U.S. Pat. No. 6,264,975, U.S. Pat. No. 5,656,256, and U.S. Pat. No. 5,292,498, each of which is incorporated herein by reference.

Bronchodiloators can also be used in combination with compounds of the present invention. These bronchodilators include, but are not limited to, β-adrenergic agonists including but not limited to epinephrine, isoproterenol, fenoterol, albutereol, terbutalin, pirbuterol, bitolterol, metaproterenol, iosetharine, salmeterol xinafoate, as well as anticholinergic agents including but not limited to ipratropium bromide, as well as compounds such as theophylline and aminophylline. These compounds may be administered in accordance with known techniques, either prior to or concurrently with the active compounds described herein.

Another aspect of the present invention is a pharmaceutical formulation, comprising sodium channel blockers and osmotically active compounds as described above in a pharmaceutically acceptable carrier (e.g., an aqueous carrier solution). In general, the sodium channel blocker is included in the composition in an amount effective to treat mucosal surfaces, such as inhibiting the reabsorption of water by mucosal surfaces, including airway and other surfaces.

The sodium channel blockers and osmotically active compounds disclosed herein may be administered to mucosal surfaces of the subject to be treated.

The dosage of the active compounds disclosed herein will vary depending on the condition being treated and the state of the subject, but generally may be from about 0.01, 0.03, 0.05, 0.1 to 1, 5, 10 or 20 mg of the pharmaceutic agent, deposited on the airway surfaces. The daily dose may be divided among one or multiple unit dose administrations. The goal is to achieve a concentration of the pharmaceutic agents on lung airway surfaces of between 10⁻⁹-10⁴ M.

In another embodiment, they are administered by administering an aerosol suspension of respirable or non-respirable particles (preferably non-respirable particles) comprised of active compound, which the subject inhales through the nose. The respirable or non-respirable particles may be liquid or solid. The quantity of active agent included may be an amount of sufficient to achieve dissolved concentrations of active agent on the airway surfaces of the subject of from about 10⁻⁹, 10⁻⁸, or 10⁻⁷ to about 10⁻³, 10⁻², 10⁻¹ moles/liter, and more preferably from about 10⁻⁹ to about 10⁻⁴ moles/liter.

Having generally described this invention, a further understanding can be obtained by reference to certain specific examples which are provided herein for purposes of illustration only and are not intended to be limiting unless otherwise specified.

### Examples

### Example 1

A specific example of an application for the dual-nebulizer technology is illustrated in the following example in which the desired co-delivery of two therapeutic agents that act synergistically is not possible due to the incompatibility of these agents in a single formulation. PS552-02 and hypertonic saline (3-7%) synergistically enhance airway surface hydration and a maximal therapeutic benefit could be achieved by the co-administration of these agents. However, due to the poor solubility of PS552-02 in NaCl solutions, these two agents cannot be formulated together. Currently, administration of PS552-02 and hypertonic saline would require two independent nebulizer treatments. However, a dual nebulizer system alleviates this issue by allowing both agents to be simultaneously delivered. Compound PS552-02 is represented by the formula:

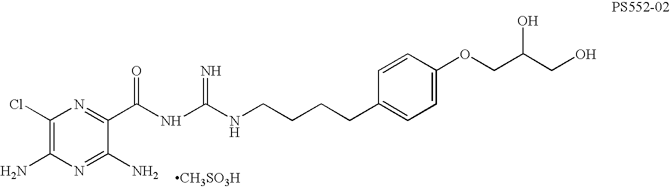

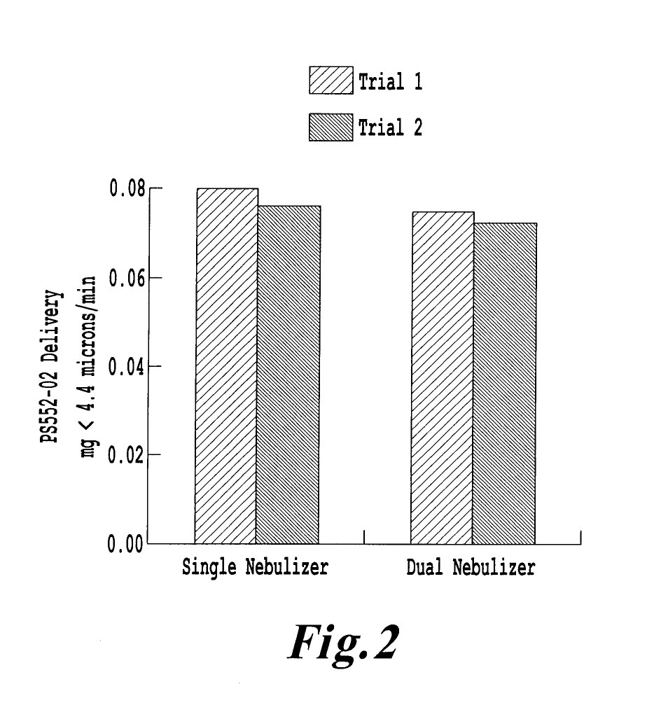

An important consideration for this nebulizer system is whether drug delivery is altered compared to what was delivered in the single nebulizer systems used in previous human and animal studies. The outputs from the dual compressor/dual nebulizer system composed of two PARI Proneb Ultra compressors and two PARI LC Star nebulizers were compared to a single PARI Proneb Ultra/PARI LC Star combination. In the dual nebulizer system, the first nebulizer contained 4 ml of 0.5 mg/ml PS552-02 in 0.12% NaCl and the other contained 4 ml of 3% NaCl. The single nebulizer contained 4 ml of 0.5 mg/ml PS552-02 in 0.12% NaCl. The aerosols from both nebulizer configurations were drawn into a Multi-Stage Liquid Impinger (Copely) at 30 L/min for 1 minute. The stages of the impinger with a cut-off less than 4.4 microns were assayed for PS552-02. FIG. 2 shows the total amount of PS552-02 less than 4.4 microns (in mg) per minute. The fine particle outputs from both systems in two studies are virtually identical, suggesting that the aerosol particles do not coalesce in the tubing (data not shown).

Obviously, numerous modifications and variations of the present invention are possible in light of the above teachings. It is therefore to be understood that within the scope of the appended claims, the invention may be practiced otherwise than as specifically described herein.

1. A device for administering two or more therapeutic agents simultaneously, comprising two or more nebulizers and a single connector linking the nebulizers to a nebulizer mouthpiece.

2. The device of claim 1, wherein the single connector is a Y-connector.

3. The device of claim 1, further comprising at least one source of compressed air connected to at least one of the nebulizers.

4. The device of claim 3, comprising one source of compressed air connected to two nebulizers.

5. The device of claim 3, wherein the source of compressed air is connected to two or more nebulizers with a Y-splitter.

6. The device of claim 3, comprising two sources of compressed air, wherein each compressor is connected to a different nebulizer.

7. The device of claim 1, wherein each nebulizer contains at least one therapeutic agent.

8. The device of claim 1, wherein each nebulizer contains a different therapeutic agent, and the therapeutic agents are incompatible in a single formulation.

9. The device of claim 1, wherein one therapeutic agent is compound PS552-02.

10. The device of claim 1, wherein one therapeutic agent is compound PS552-02 and another therapeutic agent is hypertonic saline.

11. A method of administering two or more therapeutic agents simultaneously, comprising administering the therapeutic agents simultaneously with the device of claim 1 to a subject in need thereof.

12. The method of claim 11, wherein the therapeutic agents are for treating a pulmonary disorder.

13. The method of claim 11, wherein the therapeutic agents are for treating pulmonary exposure to inhaled particles.

14. The method of claim 11, wherein the therapeutic agents are incompatible in a single formulation.

15. The method of claim 11, wherein the therapeutic agents are selected from the group consisting of DNase, Tobi, colistin, a beta agonist, a P₂Y₂ agonist, sodium chlorochromate, an osmolyte, and ENaC blockers.

16. The method of claim 11, wherein each nebulizer contains a different therapeutic agent, and the therapeutic agents are incompatible in a single formulation.

17. The method of claim 11, wherein one therapeutic agent is compound PS552-02.

18. The method of claim 11, wherein one therapeutic agent is compound PS552-02 and another therapeutic agent is hypertonic saline.
